(19) United States
(12) Patent Application Publication
(10) Pub. No.: US 2025/0352115 A1
(43) Pub. Date: Nov. 20, 2025

(54) ELECTRIC MARKOV BLANKET FOR A BRAIN-COMPUTER CRITICALITY BRIDGE

(71) Applicant: Brain Electrophysiology Laboratory Company, LLC, Eugene, OR (US)

(72) Inventors: Don M. Tucker, Eugene, OR (US); Phan Luu, Eugene, OR (US); Roman L. Shusterman, Lake Oswego, OR (US)

(21) Appl. No.: 19/246,217

(22) Filed: Jun. 23, 2025

Related U.S. Application Data
(63) Continuation-in-part of application No. 19/097,519, filed on Apr. 1, 2025.

(51) Int. Cl. A61B 5/291; A61B 5/00; A61B 5/374; A61B 5/383

(52) U.S. Cl. CPC A61B 5/291; A61B 5/374; A61B 5/383; A61B 5/7267; A61B 2562/046

(57) ABSTRACT

An electric Markov blanket (eMb) system and method for establishing a Brain-Computer Criticality Bridge (BCCB) are disclosed. Electrophysiological signals are acquired from an organism, decomposed to obtain cross-frequency coupling metrics indexing excitatory-inhibitory criticality, and assembled into a Criticality Vector that fully characterizes the organism's electric Markov blanket. The Criticality Vector may be stored, analyzed, reproduced in vivo via patterned neuromodulation, or instantiated in silico or other informatic medium to create a personal neuromorphic emulation. Embodiments include ethical-control mechanisms ensuring user sovereignty and safety. The invention enables clinical interventions, cognitive enhancement, and personal-identity preservation.

The present application is a continuation-in-part of U.S. Ser. No. 19/097,519, filed Apr. 1, 2025, which is incorporated by reference herein in its entirety.

### FIELD

The present invention relates to neurotechnology and artificial-intelligence systems for modeling, monitoring, and modulating neural criticality. More particularly, it provides methods, systems, and non-transitory computer-readable media for characterizing a living organism's Markov blanket (Mb) through electrophysiological signatures of excitatory-inhibitory (E-I) criticality, and for reproducing, therapeutically adjusting, or upgrading said signatures. By exact emulation of the person's (E-I) criticality in a personal neuromorphic emulation (PNE), a Brain Computer Criticality Bridge (BCCB) is established between the person's ongoing brain activity and the emulation of that activity in the PNE.

### BACKGROUND

Living systems maintain their integrity by predicting the adaptive context in order to adapt to it. In information terms this is a Bayesian process: minimizing surprise relative to internally-held Bayesian priors, the beliefs of the organism about the causes underlying perceived events. This minimization of surprise is implemented at the statistical boundary between organism and environment, known as the Markov blanket (Mb) (Friston, Parr et al. 2017, Friston, Parr et al. 2024, Tucker, Luu et al. 2025). At the Mb, the implicit predictions of the organism about the world meet the evidence of the world as processed by the extrinsic sensory and motor capacities of the organism. The Mb is the boundary between the mind that is always internal and the mind that more closely matches the evidence of the world.

Of course, these intrinsic and extrinsic minds should be reasonably synchronized in order to create a reasonably smooth Mb. The Mb is the information blanket that isolates, in the Markovian sense, the intrinsic predictive representations from the extrinsic corrective representations of the external-facing brain (the sensory-motor interface). It is where the predictions of active inference meet the corrections from experience in the world.

The information status of the Mb can be defined electrophysiologically by the intersection of excitatory waves underlying predictive coding and the inhibitory waves mediating error-corrections of the predictions, thereby defining the electric Markov blanket (eMb). Neurophysiological research demonstrates that critical dynamics at this information boundary of self and world emerge from cross-frequency coupling of electrophysiological recordings. For example, theta-gamma entrainment predominately indexes excitatory drive, while alpha- and beta-band rhythms index inhibitory control. Furthermore, a recent theory (Tucker, Luu, & Friston, in preparation) has suggested that memory encoding is achieved by the interference diffraction fringe created by brief (>100 ms) standing waves of (E-I) criticality (the exact balance of excitatory waves with inhibitory waves). No existing technology (i) quantifies these neurophysiological dynamics as a real-time, full-stack representation of the Mb, (ii) reconstructs or emulates them for synthetic cognition, and (iii) offers a closed-loop pathway for therapeutic neuromodulation that respects user autonomy and privacy. Furthermore, no previous technology has purported to synchronize a neurocomputation model of (E-I) criticality, the Personal Neuromorphic Emulation, that becomes fully synchronous with the person's (E-I) criticality that defines their state of conscious brain activity.

Electrophysiological characterization of the E and I balance has been described in animal studies by Gao and associates (Gao, Peterson et al. 2017). The high-frequency features of inhibitory level, I, are observed in invasive recordings in animals, including increased I in macaques under general anesthesia, and E-I ratios computed in rat hippocampal signals.

Although the EEG evidence in humans is still emerging, it is being advanced by source localization of hdEEG (>=256 channels) to the highly convoluted human cortical surface. Furthermore, recent theoretical work has explained the significance of E-I balance for cognitive function.

Within the neurocomputational model of active inference in the human brain, modeling over the last decade (Friston, et al, 2017; Tucker, et al., 2025) suggests that a living self-organizing brain (or neuromorphic emulator) minimizes variational free energy. Variational free energy is an information quantity that can be expressed by two opposing terms: (i) complexity, the divergence of current assumed information (beliefs) from prior expectations, and (ii) accuracy, the precision-weighted fit of those beliefs to actual incoming data. We express the adaptive control of criticality in neural (or neuromorphic) systems as achieved by exactly balancing two dimensionless controller gains:
Excitatory (E) control (complexity gain)—increases exploratory breadth and conceptual scope. This is the Hobsonian protoconscious predictive cognition (Hobson and Friston 2012).
Inhibitory (I) control (precision gain)—increases constraint, persistence, and prediction reliability. This neural control system provides the Hobsonian component of cognitive reality-testing.

At the neural level, excitatory (E) mechanisms are measurable in the electroencephalogram (EEG), including for example θ-γ phase-amplitude coupling (coupling between theta (˜6 Hz) and gamma (˜40 Hz) rhythms. Inhibitory (I) control is also measurable, for example, α/β burst gating. The human frequencies are well-known, although considerably different than the monkey or rodent data provided by Gao et al. In the present Variational Criticality controller, the controller continuously adjusts E and I so their ratio remains in a target band (typically 0.95-1.05); this critical set-point maximizes dynamic range and information capacity while preventing runaway excitation (σ>1.2) or pathological quiescence (σ<0.8). Thus, we treat E and I as high-level set-points in the variational controller, whereas the underlying excitatory (E) and inhibitory (I) oscillatory mechanisms serve as actuators that realize those set-points in moment-to-moment neurophysiology. These definitions anchor the preferred embodiment: hdEEG sensors estimate E, I (and branching parameter σ); the controller compares them to the critical set-point; the stimulator modulates E and/or I—through manipulating electric-Markov-blanket (eMB) fields with transcranial electrical stimulation (tES)—to maintain E and I in the optimal range for closely balanced criticality.

The meeting of E and I in a close balance appears to reflect their opponent matching: in optimal neurocognitive function the excitatory, conceptually expansive influence of E is matched by the inhibitory, conceptually focused I control. The result is the dynamic structural information of neural representations, a conceptual system that is both differentiated (I) and integrated (E) in exact proportion. Because this is a developmental process, refreshing the structural form every theta cycle (about 7 cycles per second or Hz), and capturing at least 2 beta (20 Hz) cycles in the diffraction envelope that becomes the memory representation (Tucker, Luu et al. submitted), the conceptual structure can become hierarchically organized, with multiple sequences of differentiation (I) and increasingly hierarchic integration (E).

The PNE creates its mirror of the human eMb through two-way electrical field coupling (hdEEG, high density electroencephalography, and hdtES, high density transcranial Electrical Stimulation) shown in FIG. 4. When the PNE, through use of Variational Criticality controller, maintains both its emulation and the person's brain function at criticality, then the capacity for brain-computer information exchange is enhanced, such as through long temporal correlations established at criticality (Munoz 2018) across the Electric Markov Blanket (eMb) Brain-Computer Criticality Bridge (BCCB). These long temporal correlations are predicted by criticality theory (Munoz 2018) but not fully understood at this point. The promise is that the result of the BCCB will be a extension of human consciousness in information form, achieved through the PNE's bridging between the brain and general AI, supporting an increasingly powerful neuromorphic emulation of human consciousness.

### SUMMARY

The invention, termed Electric Markov Blanket (eMb) for the Brain Computer Criticality Bridge (BCCB), provides:

1. Acquisition layer collecting high-density electrophysiology (hdEEG, MEG, invasive or non-invasive field sensors) and concurrent behavioral data.

2. Criticality-extraction engine employing Bayesian active-inference models to compute a Criticality Vector (CV) comprising theta-gamma (E) and alpha/beta-gamma (I) coupling parameters that define the Mb at millisecond resolution.

3. Self-evidencing repository storing longitudinal CVs as a neural fingerprint of the individual.

4. Reproduction module translating CVs into neuromorphic or neuromodulatory outputs (e.g., patterned tES, optogenetic drive, or silicon-based spiking-neural networks) to emulate and/or adjust the person's eMb dynamics.

5. Characterization of the interference patterns between the oscillatory regulation of excitatory influences (such as theta-gamma) and inhibitory influences (such as alpha/beta-gamma) on the cortical activity of the Markov blanket (intrinsic-extrinsic information interface).

6. Ethical control layer enforcing data sovereignty, consent management, and safe-fail constraints.

In preferred embodiments, the BCCB operates as a cloud-based platform interfacing with wearable or clinical-grade sensors and stimulators, supplying both clinical interventions (e.g., PTSD, insomnia) and personal-identity preservation within a securely-hosted Personal Neuromorphic Emulation (PNE).

### BRIEF DESCRIPTION OF THE DRAWINGS:

FIG. 1 illustrates that the Mb is co-extant with cortical surface of a person's brain, such as might be reflected by high excitatory-inhibitory criticality in the frontal lobe (brighter illustration in the frontal lobe) under certain conditions such as planning future actions. Although the Mb is defined at the layer 2-3 to 4 boundary of the cortical column, the eMb is reflected by the excitatory (e.g., theta-gamma) and inhibitory (e.g., alpha/beta) electrophysiology engaging multiple levels of the cortical column and resolved to by vhdEEG (hdEEG with Bayesian super-resolution) to the dipolar cortical macrocolumn perpendicular to the cortical surface.

FIG. 2 shows noninvasive electrodes for high density EEG, here illustrated with a 560-channel Geodesic Headweb, now implemented commercially in the 280-channel Geodesic Headweb, to mediate the physical bridge between brain and PNE computer system.

FIG. 3 illustrates a schematic overview of the eMb BCCB architecture:

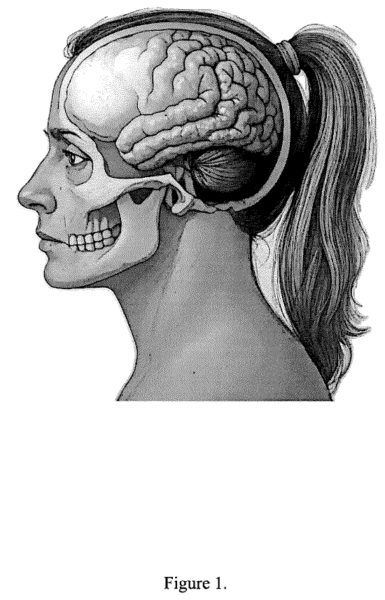

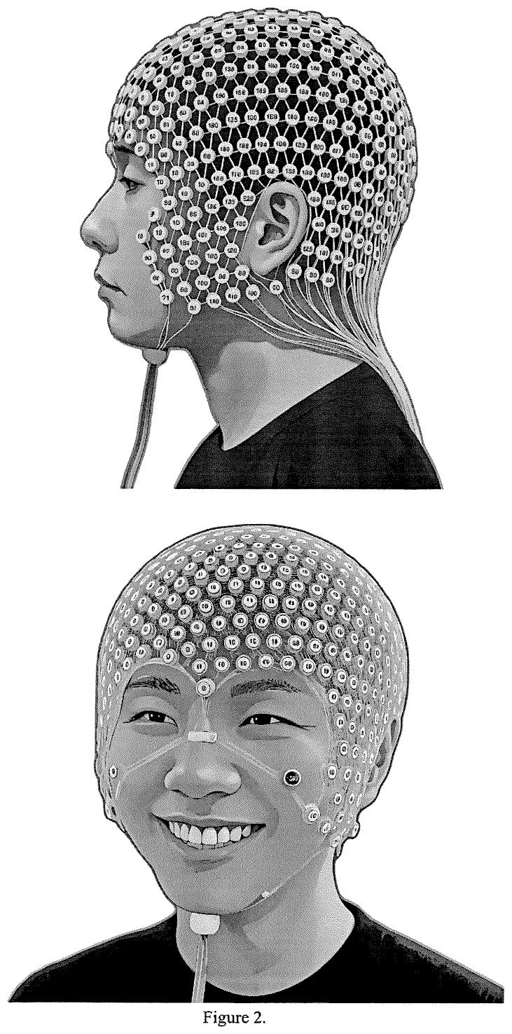

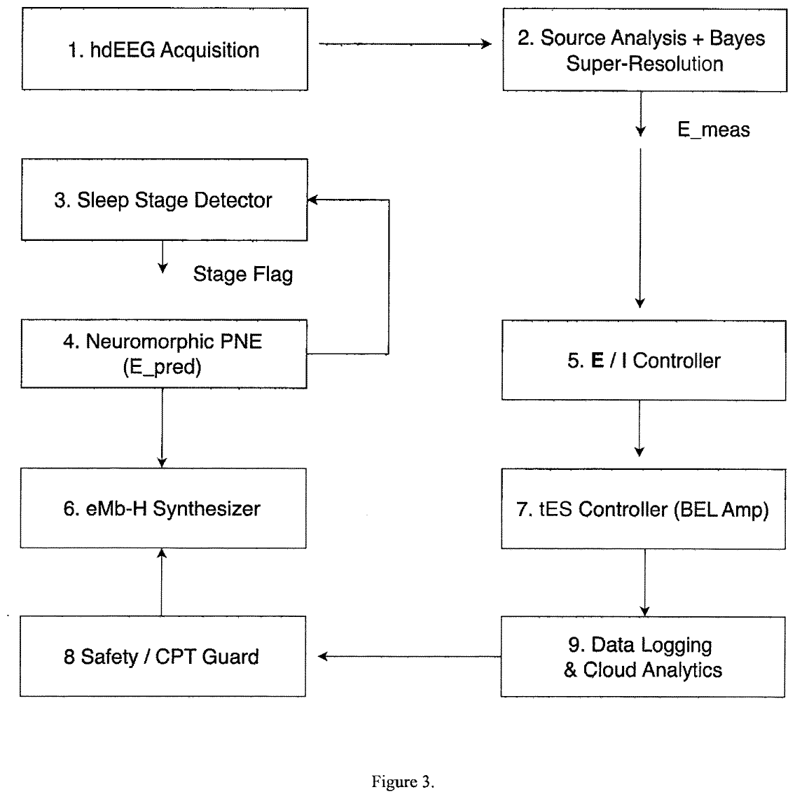

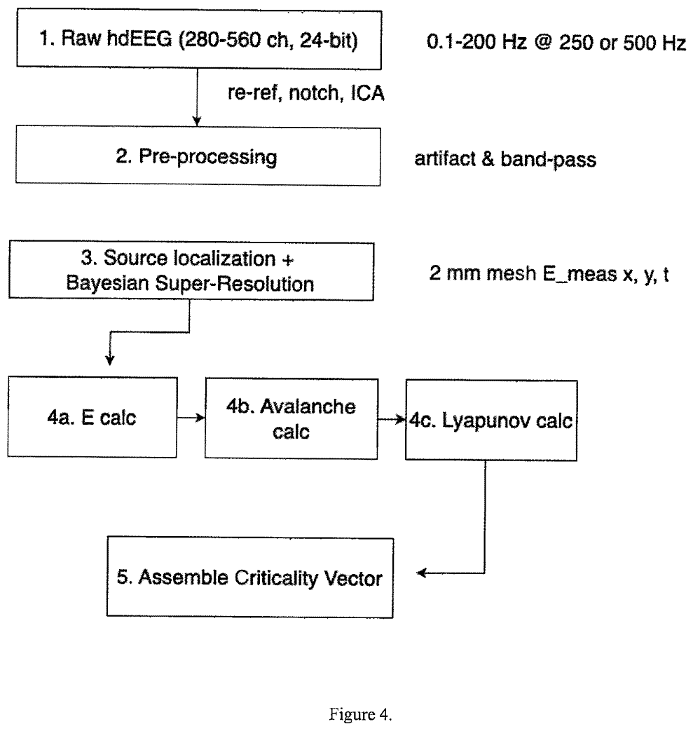

1. hd-EEG Acquisition: 280 to 560 active electrodes, 500 Hz sampling; Outputs: raw scalp-potential matrix Vscalp(t).

2. Source-Localization+Bayesian Super-Resolution: Inputs: Vscalp(t)+MRI head model; Outputs: cortical-mesh electric field Emeas(x, y, t) (2 mm res).

3. Sleep-Stage Detector: Inputs: Emeas(x, y, t); Outputs: four-state flag (Wake, NREM-N2, NREM-N3, REM).

4. Personal Neuromorphic Emulation (PNE): Neuromorphic SNN (˜10¹² synapses); Predicts next-step cortical field Epred(x, y, t+Δt).

5. E/I (and λ) Controller (Variational-Criticality Core): Inputs: Emeas, Epred, sleep-stage flag; Computes: Complexity gain E; Precision gain I; Lyapunov λ₁; Branching σ; Outputs: updated E, I and “error vector” ΔE=Epred−Emeas. Note that the E constant for Complexity scaling is equivalent to excitatory scaling of predictions in active inference, and the I constant for Accuracy scaling is equivalent to inhibitory scaling of constraints.

6. eMB-H Synthesizer (Electric Markov-Blanket Heterodyning): Inputs: ΔE, E, I, λ₁, sleep-stage flag; Solves: multi-frequency tES with high frequency (multiple KHz) carrier signals, offset by the low frequency stimulation signal applied to create a tangential current vector ΔI that minimizes radial field component and imposes desired E/I shift.

7. 280- to 560-Channel TES Array (Same Geodesic Headweb collects hdEEG and delivers hdtES): Receives ΔI (current amplitudes & phases); Generates tangential electric field along cortex (Electric Markov Blanket).

8. Safety & Ethics Gate (CPT+σ Guard): Monitors E:I band, σ, λ₁, spike-rate; Overrides or aborts stimulation if thresholds violated.

9. Data-Logging & Cloud Analytics: Stores nightly biomarkers, stimulation logs, day-time clinical scores. Feeds model-update routines and regulatory audit trail.

FIG. 4 depicts extraction of the Criticality Vector from raw electrophysiology:

| Stage | Key details with 560 ch array | Output |
|---|---|---|
| 1. Raw hdEEG | 560 active dry electrodes, 500 Hz → 280 kS/s ≈ 5.4 MB/s | Vscalp(t) |
| | stream | |
| 2. Pre-processing | Hardware notch @50/60 Hz; ICA removes blink/muscle; | Clean scalp voltages |
| | on-the-fly downmix to common average | |
| 3. Source-localisation | Multiple Spars Priors Bayesian inversion; 560-ch | Emeas(x, y, t) |
| | oversampling reduces localisation RMS error to ≤1.4 mm | |
| 4a. E calculation | Wavelet MI between 4-8 Hz carrier and 30-80 Hz γ bursts | Et |
| | vs α/β (8-20 Hz) envelope → E = MIθγ/MIαβ | |
| 4b. σ calculation | Avalanche detector on 2-mm mesh; power-law fit of size- | σt |
| | distribution exponent → σ | |
| 4c. λ₁ calculation | 2-s sliding window QR-decomposition of tangent-space | λ₁t |
| | Jacobian | |
| 5. Vector assembly | I = 1/E (precision gain) | Vcrit(t) = [E, I, σ, λ₁] |

In the “Wavelet MI” step we wavelet-transform the hdEEG signal, then compute the mutual information (MI) between two derived time-series—typically the phase of a low-frequency carrier and the amplitude (or power) of a higher-frequency burst.

Mathematically, for discrete variables X (e.g., binned θ phase) and Y (binned γ amplitude),

$$MI ( X ; Y ) = {\sum }_{x , y} p ( x , y ) {log}_{2} ( \frac{p ( x , y )}{p ( x ) p ( y )} ) bits$$

where p(x, y) is the joint probability estimated over the whole window.
MIθγ—mutual information between θ-band phase (4-8 Hz) and γ-band amplitude (30-80 Hz); high values mark strong excitatory (reference-wave in the holographic formulation) coupling→contributes to E (complexity gain).
MIαβ—mutual information between α/β phase (8-20 Hz) and β-burst amplitude; reflects inhibitory (object-wave in the holographic formulation) gating→contributes to I (precision gain).

Because MI is non-parametric and captures both linear and nonlinear dependence, it is robust for controller feedback in the Variational-Criticality framework (Tucker, Luu, & Friston 2025).

### DETAILED DESCRIPTION OF PREFERRED EMBODIMENTS:

### 1. Definitions

Markov blanket (Mb): The conditional boundary separating internal states from external states within a generative Bayesian model of an organism.

Criticality Vector (CV): A multidimensional tuple comprising electrophysiological metrics of E-I balance, such as theta-gamma phase-amplitude coupling, alpha-band inhibition index, beta-band long-range suppression, and higher-order derivatives thereof.

Personal Neuromorphic Emulation (PNE): An approximate copy of the individual's brain in an enduring computational medium capable of neural network processing.

### 2. Acquisition Layer

High-density EEG (≥280 channels, ≥1 kHz sampling) is preferred. Sensors may be complemented by ECG, galvanic skin response, behavioral logs, or environmental streams, time-locked via precision clocking.

### 3. Criticality-Extraction Engine

A variational Bayesian filter decomposes incoming signals, computes wavelet-domain metrics, and fits them to a generative model of E-I criticality. The engine outputs a real-time CV and assigns uncertainty bounds.

### 4. Self-Evidencing Repository

The CV is stored in an encrypted, append-only ledger. Differential-privacy techniques permit aggregate analytics without compromising personal data.

### 5. Reproduction Module

### 5.1 Neuromodulatory Reproduction

Pattern-adaptive transcranial electrical stimulation (tES) applies currents whose temporal envelopes mirror the target CV, achieving functional eMb alignment. Stimulation currents are phase-steered, such as with high-frequency heterodyning or “temporal interference” (Grossman, et al., 2017; Sachkov, et al., 1967) such that the induced electric field is predominantly tangential (parallel) to the cortical sheet, thereby modulating synaptic integration on the eMb itself while minimizing direct radial depolarization of cortical columns.

### 5.2 Neuromorphic Emulation

A spiking-neural network is parameterized to match the CV, enabling in-silico replication of an individual's Mb for hybrid cognition (and interface with general AI tools) through the PNE interface.

### 6. Ethical Control Layer

Smart contracts assure consent is consistent with ethical guidelines; a public-benefit algorithm vetoes stimulation patterns exceeding safety thresholds or conflicting with user-defined constraints.

### 7. Use-Case Examples of Therapeutic and Practical Applications for Improving the Excitatory-Inhibitory Balance in Sleep for Treating Clinical Disorders

The near-term applications (with existing BEL System One hdEEG/TES technology) for individualized assessment and therapy follow the known disorders of E-I neurophysiology in sleep:

| | Disordered Variational Criticality of the E-I | |
|---|---|---|
| Disorder | Balance | Noninvasive VC Therapeutic Strategy |
| Insomnia | Trouble falling or staying asleep shows up | Gentle theta or slow-wave pulses nudge the |
| | as too many fast “alert” bursts (high α/β) | ratio back toward balance, telling the cortex |
| | and a E:I ratio stuck below 0.9-the brain | “it's safe to drift.” Once E climbs and σ |
| | won't let go of daytime precision. | steadies, the natural sleep machinery can |
| | | take over without medication. |
| Major | People with depression dream early and | Brief theta bursts during REM and deeper |
| depression | often but their REM cycles lack the usual | slow-wave support restore the missing |
| | creative drive-E stays low and slow-wave | exploratory push, lifting mood circuits in the |
| | sleep is shallow. Measuring these patterns | night so daytime feelings become lighter |
| | confirms the mood circuitry is “under- | without overstimulation. |
| | exploring.” | |
| Bipolar mania / | Manic brains show the opposite: E spikes | Timed alpha pulses and slow cathodal clamps |
| hypomania | high and branching σ races past 1.2, | calm the runaway waves, pulling E and σ |
| | especially in REM and light NREM, | back into the safe band before they snowball |
| | foreshadowing racing thoughts. | into daytime mania. |
| Generalized / | Chronic worry shows up as frequent micro- | Theta stimulation plus vagus “calm” pulses |
| social / panic | arousals-tiny awakenings marked by | widen the ratio and cut the micro-arousal |
| anxiety | strong α bursts and a E:I ratio well below | count, letting broader, more flexible |
| | 0.9. The brain keeps replaying the same | predictions form overnight-so worries |
| | narrow prediction errors. | shrink by morning. |
| Drug-resistant | Before a seizure the local network turns | Slow, tangential cathodal pulses drain just |
| focal epilepsy | super-critical: σ climbs and spike-rate soars | enough excitatory drive to bring σ back to |
| | during stage-2 sleep. Detecting that | 1.0, often stopping the seizure before it starts |
| | pattern gives an early warning minutes | and reducing next-day fatigue. |
| | ahead. | |

More specifically, the following protocol embodiments illustrate how the eMb BCCB, governed by the variational-criticality (VC) optimizer, can be configured to assess and treat the major sleep-linked neuropsychiatric disorders recognized in the US Psychiatry Diagnostic and Statistical Manual (DSM-5), including epilepsy. Each protocol employs the same three-step logic:
1. Baseline characterization—overnight hdEEG establishes disorder-specific signatures in the criticality vector V=[E, I, σ, λ₁].
2. Stage-gated intervention—the E/I controller activates tangential tES bursts (eMB-Heterodyning) only when sleep stage and criticality criteria match the disorder template.
3. Adaptive convergence—stimulation parameters self-adjust until V re-enters a predefined “therapeutic band,” after which the burst train is tapered.

Unless otherwise stated, stimulation is delivered through a 280-channel (or next-generation 560-channel) hdEEG/TES Geodesic Headweb, supported by the BEL System One and FLOW cloud, current-steered to maximize the electric Markov blanket's tangential component (>70% of total field energy).

A. Insomnia (Sleep-Onset and Maintenance):

| Phase | Baseline finding | Intervention | Target shift |
|---|---|---|---|
| Sleep onset (N1 delay) | E:I persistently <0.9; | Theta-burst TES (6 Hz, | E ↑ ≈ +0.05 |
| | α/β burst density >15 | 0.75 mA, 5 s ON / 5 s | |
| | min⁻¹ | OFF) delivered fronto- | |
| | | parietally until E:I ≥0.95 | |
| | | or sleep scores N2 | |
| Maintenance (fragmented | σ falls <0.8 after arousals | Slow-wave tACS (0.75 | σ ↑ → 0.9-1.0 |
| N2/N3) | | Hz, 1.5 mA, 5-cycle | |
| | | trains) phase-locked to | |
| | | up-state | |

B. Major Depressive Disorder:

| Sleep stage | Baseline finding | Intervention | Stop rule |
|---|---|---|---|
| REM (excess density, low | E:I ≤0.85 for ≥20 s | Theta-burst TES (6 Hz, 1 | E:I stable 0.95-1.05 |
| E) | bursts | mA) beginning 5 s after | for ≥5 min |
| | | REM onset; repeat every | |
| | | 30 s until E:I ≥0.95 | |
| N3 (reduced slow waves) | I dips <0.75, σ ≈ 0.9 | 0.75 Hz tACS (1.5 mA, | I → 0.85-1.0, σ → 1.0 |
| | | fronto-central) during | |
| | | first two N3 cycles | |

C. Bipolar Mania / Hypomania:

| Sleep stage | Baseline finding | Intervention | Stop rule |
|---|---|---|---|
| REM (E spikes >1.10) | E:I ≥1.10 for ≥3 min | Alpha-burst TES (10 Hz, | E:I ≤1.05 or σ <1.2 |
| | | 1 mA, 30 s ON / 30 s | |
| | | OFF) targeted F3-F4 | |
| Stage-2 NREM | σ surges >1.20 pre-ictal- | 0.5 Hz cathodal pulses | σ → 0.95-1.05 |
| | like | (100 ms, 2 mA peak) to | |
| | | clamp σ | |

D. Generalized Anxiety Disorder / Social Anxiety / Panic:

| Sleep stage | Baseline finding | Intervention | Target |
|---|---|---|---|
| N2 (α/β micro-arousals) | E:I ≤0.88; α bursts >12 | Theta burst TES (6 Hz, | E:I ≥0.95; α burst |
| | min⁻¹ | 1.2 mA, 5 s ON / 10 s | density <8 min⁻¹ |
| | | OFF) + auricular vagus | |
| | | stimulation (500 ms, 30 | |
| | | Hz) when α bursts exceed | |
| | | threshold | |
| REM (attenuated θ-γ) | MIθγ <0.15 bits | 6 Hz theta-tACS (0.75 | E:I maintained 0.95- |
| | | mA) delivered midline | 1.05 |
| | | until MIθγ ≥0.22 | |

E. Drug-Resistant Focal Epilepsy:

| Sleep stage | Baseline finding | Intervention | Stop rule |
|---|---|---|---|
| Stage-2 NREM | σ ≥1.25 or spike rate > | Slow-pulsed cathodal TES | σ ≤1.1 and spike-rate |
| | baseline +30% | (0.5 Hz, 100 ms, 2 mA) | ↓ ≥30% |
| | | tangential to | |
| | | epileptogenic focus | |
| REM | E oscillations suppressed | No stimulation unless E:I | — |
| | (protect memory) | falls <0.9 (then 6 Hz θ- | |
| | | burst) | |

Controller logic (common to all disorders):
1. Continuous monitoring of V=[E, I, σ, λ₁] at 2-s resolution.
2. Stage gate: interventions allowed only when stage detector=relevant stage (as specified in tables).
3. Error computation: E_gain=|E−E_target|+|I−I_target|+|σ−1|.
4. Pulse scheduler: selects frequency, amplitude, train length proportional to E_gain (bounded by 2 mA/contact and Σ|I|≤I_max).
5. Safety override: stimulation disabled if λ₁<−0.05 s⁻¹ (coma-risk) or >0.05 s⁻¹ (seizure-risk) for >10 s.

Expected therapeutic trajectory:
Week 0-1—Baseline nights only; classifier builds disorder signature.
Week 1-3—Adaptive intervention; E/I and σ converge toward therapeutic band during target stages.
Week 4+—Maintenance phase; stimulation delivered only on relapse nights (<30% duty cycle), preserving battery life and neuroplastic balance.

### 8. Hardware Variants

EMBAR may be instantiated on custom neuromorphic ASICs, FPGA accelerators, or general-purpose GPU clusters; low-power edge devices are contemplated.

### 9. Software Implementation

Software is delivered as containerized micro-services communicating via gRPC. A domain-specific language (DSL) specifies eMb queries and stimulation recipes.

In certain optional embodiments, the low-frequency electrophysiological excitatory sheet (‘reference wave’) and the high-frequency inhibitory packets (‘object wave’), such as theorized in the NREM and REM components of active inference in sleep (Tucker, Luu et al. 2025) may form interference patterns analogous to Pribram's (1970) holonomic model, potentially serving as a distributed memory substrate. Phase-controlled eMB TES re-contacts (“re-illumines” in the holography analogy) the inhibitory electrical reference wave to reconstruct the excitatory object-wave assembly, enabling phase-specific memory retrieval. However, practical operation of the present controller does not depend on this holonomic hypothesis of the eMb information storage dynamics, which requires further scientific validation.

### REFERENCES CITED

Friston, K. J., Parr, T., & de Vries, B. 2017. The graphical brain: Belief propagation and active inference. Network Neuroscience, 1(4), 381-414.
Friston, K. J., Parr, T., Heins, C., Constant, A., Friedman, D., Isomura, T., . . . Frith, C. D. 2024. Federated inference and belief sharing. Neuroscience & Biobehavioral Reviews, 156, 105500.
Gao, R., Peterson, E. J., & Voytek, B. 2017. Inferring synaptic excitation/inhibition balance from field potentials. Neuroimage, 158, 70-78.
Grossberg, S. 1980. How does a brain build a cognitive code? Psychological Review, 87(0), 1-51.
Grossman, N., Bono, D., Dedic, N., Kodandaramaiah, S. B., Rudenko, A., Suk, H. J., . . . Boyden, E. S. 2017. Noninvasive Deep Brain Stimulation via Temporally Interfering Electric Fields. Cell, 169(6), 1029-1041 e1016.
Pribram, K. H. (1977). Holonomy and Structure in the Organization of Perception. Paper presented at the Images, Perception, and Knowledge: Papers Deriving from and Related to the Philosophy of Science Workshop at Ontario, Canada, May 1974.
Sachkov, V.I., Liventsev, N.M., Kuzin, M.I., Zhukovskii, V.D., 1967. Experiences with combined electronarcosis with interference currents in clinical surgery, In: Wagender, F.M., Schuy, S. (Eds.), Electrotherapeutic Sleep and Electroanesthesia. Medica Foundation, pp. 321-326.
Tucker, D. M. 2007. Mind From Body: Experience From Neural Structure. New York: Oxford University Press.
Tucker, D. M., Luu, P., & Friston, K. J. 2025. Adaptive consolidation of active inference: excitatory and inhibitory mechanisms for organizing feedforward and feedback memory systems in sleep. Cerebral Cortex, 35(5), bhaf122.

1. A method for characterizing a Markov blanket of a living organism, comprising:
(a) acquiring electrophysiological signals representing neural activity;
(b) computing a Criticality Vector comprising measures of theta gamma, alpha, and beta coupling indicative of excitatory-inhibitory criticality; and
(c) storing said Criticality Vector in association with the organism.

2. The method of claim 1, further comprising fitting a Bayesian generative model to said signals to minimize free energy and update priors representing the organism's self-evidencing.

3. The method of claim 1 or 2, further comprising reproducing the Criticality Vector by applying a patterned neuromodulatory stimulus to the organism.

4. The method of claim 3, wherein the stimulus comprises transcranial electrical currents phase-locked to said theta-gamma and alpha/beta oscillations.

5. The method of any of claims 1-4, wherein said electrophysiological signals are acquired using ≥130-channel EEG at ≥250 Hz sampling.

6. A system comprising: (i) a sensor array configured to acquire electrophysiological data;
(ii) a processor executing instructions to compute a Criticality Vector; (iii) a non-volatile memory storing said vector; and (iv) an actuator configured to reproduce said vector via neuromodulation.

7. The system of claim 6, wherein the processor further executes an ethical-control module preventing stimulation that violates predefined safety constraints.

8. The system of claim 6 or 7, wherein the actuator is a multi-electrode tES device delivering current waveforms parameterized by the Criticality Vector.

9. The system of any of claims 6-8, further comprising a neuromorphic computing unit configured to emulate the organism's Markov blanket based on the Criticality Vector.

10. A non-transitory computer-readable medium storing instructions that, when executed, perform the method of any of claims 1-4.

11. The method of claim 3, wherein reproducing the Criticality Vector is performed in a physically separate neuromorphic device to create a functional replica of the organism's Markov blanket.

12. The system of claim 9, wherein the neuromorphic computing unit comprises spiking-neural-network cores operating at sub-threshold leakage currents ≤1 pJ per synaptic event.

13. The method of claim 1, further comprising generating a longitudinal record of Criticality Vectors and using machine learning to forecast future Mb states.

14. The system of claim 6, wherein the processor provides an application programming interface (API) for third-party software to query the Criticality Vector under encrypted differential-privacy constraints.

15. The method of claim 3, wherein the neuromodulatory stimulus is titrated according to a real-time comparison between an observed Criticality Vector and a target Criticality Vector.

16. The method of claim 3, wherein the localization of hdEEG activity to the cortical surface is achieved through a Bayesian super-resolution algorithm such as that disclosed in U.S. application Ser. No. 19/097,519.

17. The method of claim 3, wherein the synchronization of power spectra follows the methods Gao and associates (Gao, Peterson et al. 2017).
